(19) United States
(12) Patent Application Publication
(10) Pub. No.: US 2025/0367272 A1
(43) Pub. Date: Dec. 4, 2025

(54) HIRUDIN FORMULA COMPOSITION WITH ANTICOAGULANT AND HYPOLIPIDEMIC EFFECTS AND PREPARATION METHOD THEREOF

(71) Applicant: NANO BIOLOGY LIMITED, RENTON, WA (US)

(72) Inventors: Xinxin LU, Ankang (CN); Likang CHEN, Ankang (CN); Yukang ZHANG, Ankang (CN)

(73) Assignee: NANO BIOLOGY LIMITED, RENTON, WA (US)

(21) Appl. No.: 18/679,708

(22) Filed: May 31, 2024

(51) Int. Cl. A61K 38/58; A61K 9/48; A61K 31/26; A61K 31/352; A61K 36/062; A61K 36/07; A61K 36/258; A61K 38/48; A61P 3/06; A61P 7/04; C07K 1/36

(52) U.S. Cl. CPC A61K 38/58; A61K 9/48; A61K 31/26; A61K 31/352; A61K 36/062; A61K 36/071; A61K 36/258; A61K 38/482; A61P 3/06; A61P 7/04; C07K 1/36; C12Y 304/21062; A61K 2236/15; A61K 2236/331; A61K 2236/51

(57) ABSTRACT

The disclosure relates to the technical field of dietary nutritional supplements, in particular to a hirudin formula composition with anticoagulant and hypolipidemic effects and a preparation method thereof. Technical problem: the hirudin formula composition with anticoagulant and hypolipidemic effects and the preparation method thereof are intended to solve the technical problems that dietary nutritional supplements in the prior art cannot achieve anticoagulant and hypolipidemic effects. Technical solution: a hirudin formula composition with anticoagulant and hypolipidemic effects includes the following components by weight: 50 mg to 500 mg of hirudin polypeptide, 10 mg to 100 mg of red yeast rice, 10 mg to 100 mg of nattokinase, 10 mg to 100 mg of sulforaphane, 10 mg to 100 mg of agaricus blazei murill, 10 mg to 100 mg of quercetin, 10 mg to 300 mg of hirudin, and 50 mg to 300 mg of panax notoginseng extract.

### TECHNICAL FIELD

The disclosure relates to the technical field of dietary nutritional supplements, in particular to a hirudin formula composition with anticoagulant and hypolipidemic effects and a preparation method thereof.

### BACKGROUND

Coagulation disorders are of a large category. The coagulation disorders may cause the failure of the human body to control bleeding effectively, which means that the body fails to stop or limit the amount of bleeding even if it is injured or traumatized. In some cases, the coagulation disorders may lead to excessive clotting of blood in blood vessels, forming blood clots, which may block blood vessels, affect blood circulation and cause diseases such as stroke and myocardial infarction. Due to the coagulation disorders, the body fails to control the bleeding at a site of an injury, which leads to the formation of ecchymosis and congestion, resulting in local swelling and pain. Hyperlipidemia may lead to a variety of health problems, including atherosclerosis, coronary heart disease, high blood pressure, stroke, peripheral artery disease, etc. However, the treatments of coagulation disorders and hyperlipidemia by the prior art mainly include drug therapy, dietary regulation and exercise. However, the drug treatment will lead to an increase in the body's resistance to drugs, resulting in secondary drug failure. At the same time, frequent medication will cause irritations to the digestive system, such as gastric mucosal erosion, corrosion, as well as acid reflux, heartburn, nausea, vomiting and other conditions. Most drugs have to be metabolized by the liver and kidney, and may increase the loads on the liver and kidney if they are used for a long time or in a large dosage, thereby causing liver and kidney damages; and some drugs also have liver and kidney toxicity, and may lead to liver and kidney failure resulting from improper medication. Some patients may have allergic reactions after taking drugs, such as skin itching, rash and other manifestations in mild cases, and anaphylactic shock and even life-threatening in severe cases. However, ordinary dietary nutritional supplements cannot achieve anticoagulant and hypolipidemic effects.

### SUMMARY

In view of the defects of the prior art, an object of the disclosure is to provide a hirudin formula composition with anticoagulant and hypolipidemic effects and a preparation method thereof. The hirudin formula composition with anticoagulant and hypolipidemic effects and the preparation method thereof are intended to solve the technical problems that dietary nutritional supplements in the prior art cannot achieve anticoagulant and hypolipidemic effects.

A technical solution of the disclosure is as follows: a hirudin formula composition with anticoagulant and hypolipidemic effects includes the following components by weight:

| | |
|---|---|
| hirudin polypeptide | 50 mg to 500 mg; |
| red yeast rice | 10 mg to 100 mg; |
| nattokinase | 10 mg to 100 mg; |
| sulforaphane | 10 mg to 100 mg; |
| agaricus blazei murill | 10 mg to 100 mg; |
| quercetin | 10 mg to 100 mg; |
| hirudin | 10 mg to 300 mg; and |
| panax notoginseng extract | 50 mg to 300 mg. |

Preferably, a preparation method of the hirudin polypeptide includes: homogenizing fresh leeches in water, wherein a weight ratio of the water to the leeches is (1 to 3): 1; centrifuging the homogenate at 10000 r/min for 10 min, and then filtering a precipitate; mixing the precipitate and water in a reactor in a dosage ratio of (3 to 15 g): 100 ml; controlling a pH value to 7.0 to 10.0, and then adding alkaline protease; reacting for 1 to 5 h at a reaction temperature of 40 to 70° C. and an oscillation speed of 100 to 300 rpm; and freeze-drying to obtain the hirudin polypeptide.

Preferably, a preparation method of the nattokinase includes: inoculating a strain, that is isolated and screened from natto, into a liquid medium, culturing at a constant temperature of 37° C., and centrifuging at 4000 rpm for 30 min; taking a supernatant, i.e., a crude enzyme solution, and removing impure proteins and polysaccharides from the crude enzyme solution; taking a supernatant, adding ammonium sulfate slowly to the supernatant to a saturation of 25%, keeping at low temperature overnight, and centrifuging at 4000 rpm for 30 min to remove impure proteins; then taking a supernatant, adding ammonium sulfate to the supernatant until a final saturation is 60%, keeping at low temperature overnight, and centrifuging at 4000 rpm for 30 min to remove polysaccharides; and collecting a precipitate, dissolving the precipitate in a buffer solution, desalting by dialysis, removing ammonium sulfate, and purifying a sample to obtain the nattokinase.

Preferably, the method for removing the impure proteins and the polysaccharides from the crude enzyme solution is performed by fractional precipitation of ammonium sulfate, and the purification process includes column chromatography, dialysis, and membrane separation.

Preferably, an extraction step of the sulforaphane includes: taking cruciferous vegetables, adding 8 to 15 times the amount of water, and performing enzymatic hydrolysis by heating in a water bath at a temperature of 30° C. to 45° C. for 2 to 6 h; and then, adding methanol for ultrasonic extraction, centrifuging the obtained extract to remove a supernatant, and freeze-drying to obtain the sulforaphane.

Preferably, a preparation method of the quercetin includes: crushing oak barks, extracting with 8 to 12 times the amount of ethanol or methanol, and then filtering, concentrating and crystallizing to obtain a crude quercetin product; and finally performing further separation and purification to obtain the high-purity quercetin.

Preferably, an extraction step of the hirudin includes: taking fresh leeches, washing impurities and bacteria on the surfaces of the fresh leeches, and chopping the fresh leeches into fine particles; putting the chopped leeches into 10 to 15 times the amount of purified water, and stirring them in a thermostatic shaker for 1 h; centrifuging the stirred liquid, collecting a precipitate, and washing the precipitate with purified water; repeating this step for three times until a pure leech extract is collected; and drying the leech extract through a nitrogen sampler to obtain a dried hirudin polypeptide.

Preferably, a preparation method of the panax notoginseng extract includes: crushing panax notoginseng, and pouring into an extraction tank; extracting the panax notoginseng with clear water according to a mass ratio of 1:(10 to 15); retaining and concentrating a filtrate; spray-drying concentrated thick paste; and then crushing and sieving to obtain the panax notoginseng extract.

A preparation method of a hirudin formula composition with anticoagulant and hypolipidemic effects includes the hirudin formula composition with anticoagulant and hypolipidemic effects and the following steps:
step 1: weighing formulated amounts of hirudin polypeptide, red yeast rice, nattokinase, sulforaphane, agaricus blazei murill, quercetin, hirudin and panax notoginseng extract;
step 2: adding the hirudin polypeptide, the red yeast rice, the nattokinase, the sulforaphane, the agaricus blazei murill, the quercetin, the hirudin and the panax notoginseng extract into a mixer, and mixing well uniformly; and
step 3: preparing the mixture in step 2 into capsules.

The disclosure has the following beneficial effects: the hirudin formula composition contains a variety of active ingredients, has a strong inhibitory effect on thrombin and the effects of lowering blood lipids and blood pressure and accelerating blood circulation, and can prevent cardiovascular and cerebrovascular diseases, make up for the problem of insufficient intake in normal diet, and reduce the side effects caused by drug treatment. Hirudin is the most active and most studied ingredient among a variety of active ingredients that have been extracted from leeches and their salivary glands, and is a small molecule protein composed of 65-66 amino acids. The leeches contain rich hirudin, which has a strong inhibitory effect on thrombin and is the strongest natural specific inhibitor for thrombin found so far. The red yeast rice has the effects of reducing total cholesterol, low-density lipoprotein cholesterol, serum triglyceride and atherosclerosis indexes, and increasing high-density lipoprotein cholesterol, which is of great significance for the prevention and treatment of cardiovascular and cerebrovascular diseases, and lowering of blood pressure and blood lipid. The nattokinase has the effects of reducing fibrinogen, dissolving thrombosis, resisting platelet aggregation, reducing blood viscosity, and improving blood circulation. The sulforaphane can also promote the production of protective enzymes in human cells, enhance the activity of detoxification enzymes in the body, and effectively eliminate harmful substances in the human body, has a certain effect of promoting the detoxification of the liver and lungs, and can also promote the metabolism of the body. The agaricus blazei murill has the effects of promoting hematopoiesis, protecting the liver and kidney, and enhancing physical fitness. The quercetin can lower blood pressure and cholesterol, and thus reduce the risk of cardiovascular diseases. At the same time, the quercetin can also improve a vascular endothelial function and prevent atherosclerosis. The panax notoginseng extract has a protective effect on blood vessels.

### DETAILED DESCRIPTION OF THE EMBODIMENTS

The disclosure will be further described below in conjunction with the examples.

### Example 1

The disclosure provides an example: a hirudin formula composition with anticoagulant and hypolipidemic effects includes the following components by weight:

| | |
|---|---|
| hirudin polypeptide | 200 mg; |
| red yeast rice | 100 mg; |
| nattokinase | 50 mg; |
| sulforaphane | 50 mg; |
| agaricus blazei murill | 100 mg; |
| quercetin | 100 mg; |
| hirudin | 200 mg; and |
| panax notoginseng extract | 200 mg. |

Preferably, a preparation method of the hirudin polypeptide includes: homogenizing fresh leeches in water, wherein a weight ratio of the water to the leeches is 1:1; centrifuging the homogenate at 10000 r/min for 10 min, and then filtering a precipitate; mixing the precipitate and water in a reactor in a dosage ratio of 5 g:100 ml; controlling a pH value to 7.0 to 10.0, and then adding alkaline protease; reacting for 2 h at a reaction temperature of 45° C. and an oscillation speed of 100 rpm; and freeze-drying to obtain the hirudin polypeptide.

Preferably, a preparation method of the nattokinase includes: inoculating a strain, that is isolated and screened from natto, into a liquid medium, culturing at a constant temperature of 37° C., and centrifuging at 4000 rpm for 30 min; taking a supernatant, i.e., a crude enzyme solution, and removing impure proteins and polysaccharides from the crude enzyme solution; taking a supernatant, adding ammonium sulfate slowly to the supernatant to a saturation of 25%, keeping at low temperature overnight, and centrifuging at 4000 rpm for 30 min to remove impure proteins; then taking a supernatant, adding ammonium sulfate to the supernatant until a final saturation is 60%, keeping at low temperature overnight, and centrifuging at 4000 rpm for 30 min to remove polysaccharides; and collecting a precipitate, dissolving the precipitate in a buffer solution, desalting by dialysis, removing ammonium sulfate, and purifying a sample to obtain the nattokinase.

Preferably, the method for removing the impure proteins and the polysaccharides from the crude enzyme solution is performed by fractional precipitation of ammonium sulfate, and the purification process includes column chromatography, dialysis, and membrane separation.

Preferably, an extraction step of the sulforaphane includes: taking cruciferous vegetables, adding 8 times the amount of water, and performing enzymatic hydrolysis by heating in a water bath at a temperature of 30° C. for 6 h; and then, adding methanol for ultrasonic extraction, centrifuging the obtained extract to remove a supernatant, and freeze-drying to obtain the sulforaphane.

Preferably, a preparation method of the quercetin includes: crushing oak barks, extracting with 8 times the amount of ethanol, and then filtering, concentrating and crystallizing to obtain a crude quercetin product; and finally performing further separation and purification to obtain the high-purity quercetin.

Preferably, an extraction step of the hirudin includes: taking fresh leeches, washing impurities and bacteria on the surfaces of the fresh leeches, and chopping the fresh leeches into fine particles; putting the chopped leeches into 10 times the amount of purified water, and stirring them in a thermostatic shaker for 1 h; centrifuging the stirred liquid, collecting a precipitate, and washing the precipitate with purified water; repeating this step for three times until a pure leech extract is collected; and drying the leech extract through a nitrogen sampler to obtain a dried hirudin polypeptide.

Preferably, a preparation method of the panax notoginseng extract includes: crushing panax notoginseng, and pouring into an extraction tank; extracting the panax notoginseng with clear water according to a mass ratio of 1:10; retaining and concentrating a filtrate; spray-drying concentrated thick paste; and then crushing and sieving to obtain the panax notoginseng extract.

A preparation method of a hirudin formula composition with anticoagulant and hypolipidemic effects includes the hirudin formula composition with anticoagulant and hypolipidemic effects and the following steps:
step 1: weighing formulated amounts of hirudin polypeptide, red yeast rice, nattokinase, sulforaphane, agaricus blazei murill, quercetin, hirudin and panax notoginseng extract;
step 2: adding the hirudin polypeptide, the red yeast rice, the nattokinase, the sulforaphane, the agaricus blazei murill, the quercetin, the hirudin and the panax notoginseng extract into a mixer, and mixing well uniformly; and
step 3: preparing the mixture in step 2 into capsules.

### Example 2

The disclosure provides an example: a hirudin formula composition with anticoagulant and hypolipidemic effects includes the following components by weight:

| | |
|---|---|
| hirudin polypeptide | 300 mg; |
| red yeast rice | 50 mg; |
| nattokinase | 100 mg; |
| sulforaphane | 50 mg; |
| agaricus blazei murill | 50 mg; |
| quercetin | 50 mg; |
| hirudin | 300 mg; and |
| panax notoginseng extract | 100 mg. |

Preferably, a preparation method of the hirudin polypeptide includes: homogenizing fresh leeches in water, wherein a weight ratio of the water to the leeches is 3:1; centrifuging the homogenate at 10000 r/min for 10 min, and then filtering a precipitate; mixing the precipitate and water in a reactor in a dosage ratio of 8 g:100 ml; controlling a pH value to 7.0 to 10.0, and then adding alkaline protease; reacting for 3 h at a reaction temperature of 60° C. and an oscillation speed of 300 rpm; and freeze-drying to obtain the hirudin polypeptide.

Preferably, a preparation method of the nattokinase includes: inoculating a strain, that is isolated and screened from natto, into a liquid medium, culturing at a constant temperature of 37° C., and centrifuging at 4000 rpm for 30 min; taking a supernatant, i.e., a crude enzyme solution, and removing impure proteins and polysaccharides from the crude enzyme solution; taking a supernatant, adding ammonium sulfate slowly to the supernatant to a saturation of 25%, keeping at low temperature overnight, and centrifuging at 4000 rpm for 30 min to remove impure proteins; then taking a supernatant, adding ammonium sulfate to the supernatant until a final saturation is 60%, keeping at low temperature overnight, and centrifuging at 4000 rpm for 30 min to remove polysaccharides; and collecting a precipitate, dissolving the precipitate in a buffer solution, desalting by dialysis, removing ammonium sulfate, and purifying a sample to obtain the nattokinase.

Preferably, the method for removing the impure proteins and the polysaccharides from the crude enzyme solution is performed by fractional precipitation of ammonium sulfate, and the purification process includes column chromatography, dialysis, and membrane separation.

Preferably, an extraction step of the sulforaphane includes: taking cruciferous vegetables, adding 10 times the amount of water, and performing enzymatic hydrolysis by heating in a water bath at a temperature of 35° C. for 4 h; and then, adding methanol for ultrasonic extraction, centrifuging the obtained extract to remove a supernatant, and freeze-drying to obtain the sulforaphane.

Preferably, a preparation method of the quercetin includes: crushing oak barks, extracting with 12 times the amount of methanol, and then filtering, concentrating and crystallizing to obtain a crude quercetin product; and finally performing further separation and purification to obtain the high-purity quercetin.

Preferably, an extraction step of the hirudin includes: taking fresh leeches, washing impurities and bacteria on the surfaces of the fresh leeches, and chopping the fresh leeches into fine particles; putting the chopped leeches into 13 times the amount of purified water, and stirring them in a thermostatic shaker for 1 h; centrifuging the stirred liquid, collecting a precipitate, and washing the precipitate with purified water; repeating this step for three times until a pure leech extract is collected; and drying the leech extract through a nitrogen sampler to obtain a dried hirudin polypeptide.

Preferably, a preparation method of the panax notoginseng extract includes: crushing panax notoginseng, and pouring into an extraction tank; extracting the panax notoginseng with clear water according to a mass ratio of 1:11; retaining and concentrating a filtrate; spray-drying concentrated thick paste; and then crushing and sieving to obtain the panax notoginseng extract.

A preparation method of a hirudin formula composition with anticoagulant and hypolipidemic effects includes the hirudin formula composition with anticoagulant and hypolipidemic effects and the following steps:
step 1: weighing formulated amounts of hirudin polypeptide, red yeast rice, nattokinase, sulforaphane, agaricus blazei murill, quercetin, hirudin and panax notoginseng extract;
step 2: adding the hirudin polypeptide, the red yeast rice, the nattokinase, the sulforaphane, the agaricus blazei murill, the quercetin, the hirudin and the panax notoginseng extract into a mixer, and mixing well uniformly; and
step 3: preparing the mixture in step 2 into capsules.

### Example 3

The disclosure provides an example: a hirudin formula composition with anticoagulant and hypolipidemic effects includes the following components by weight:

| | |
|---|---|
| hirudin polypeptide | 400 mg; |
| red yeast rice | 50 mg; |
| nattokinase | 50 mg; |
| sulforaphane | 50 mg; |
| agaricus blazei murill | 50 mg; |
| quercetin | 100 mg; |
| hirudin | 100 mg; and |
| panax notoginseng extract | 200 mg. |

Preferably, a preparation method of the hirudin polypeptide includes: homogenizing fresh leeches in water, wherein a weight ratio of the water to the leeches is 2:1; centrifuging the homogenate at 10000 r/min for 10 min, and then filtering a precipitate; mixing the precipitate and water in a reactor in a dosage ratio of 9 g:100 ml; controlling a pH value to 7.0 to 10.0, and then adding alkaline protease; reacting for 5 h at a reaction temperature of 50° C. and an oscillation speed of 200 rpm; and freeze-drying to obtain the hirudin polypeptide.

Preferably, a preparation method of the nattokinase includes: inoculating a strain, that is isolated and screened from natto, into a liquid medium, culturing at a constant temperature of 37° C., and centrifuging at 4000 rpm for 30 min; taking a supernatant, i.e., a crude enzyme solution, and removing impure proteins and polysaccharides from the crude enzyme solution; taking a supernatant, adding ammonium sulfate slowly to the supernatant to a saturation of 25%, keeping at low temperature overnight, and centrifuging at 4000 rpm for 30 min to remove impure proteins; then taking a supernatant, adding ammonium sulfate to the supernatant until a final saturation is 60%, keeping at low temperature overnight, and centrifuging at 4000 rpm for 30 min to remove polysaccharides; and collecting a precipitate, dissolving the precipitate in a buffer solution, desalting by dialysis, removing ammonium sulfate, and purifying a sample to obtain the nattokinase.

Preferably, the method for removing the impure proteins and the polysaccharides from the crude enzyme solution is performed by fractional precipitation of ammonium sulfate, and the purification process includes column chromatography, dialysis, and membrane separation.

Preferably, an extraction step of the sulforaphane includes: taking cruciferous vegetables, adding 14 times the amount of water, and performing enzymatic hydrolysis by heating in a water bath at a temperature of 42° C. for 2 h; and then, adding methanol for ultrasonic extraction, centrifuging the obtained extract to remove a supernatant, and freeze-drying to obtain the sulforaphane.

Preferably, a preparation method of the quercetin includes: crushing oak barks, extracting with 10 times the amount of ethanol, and then filtering, concentrating and crystallizing to obtain a crude quercetin product; and finally performing further separation and purification to obtain the high-purity quercetin.

Preferably, an extraction step of the hirudin includes: taking fresh leeches, washing impurities and bacteria on the surfaces of the fresh leeches, and chopping the fresh leeches into fine particles; putting the chopped leeches into 15 times the amount of purified water, and stirring them in a thermostatic shaker for 1 h; centrifuging the stirred liquid, collecting a precipitate, and washing the precipitate with purified water; repeating this step for three times until a pure leech extract is collected; and drying the leech extract through a nitrogen sampler to obtain a dried hirudin polypeptide.

Preferably, a preparation method of the panax notoginseng extract includes: crushing panax notoginseng, and pouring into an extraction tank; extracting the panax notoginseng with clear water according to a mass ratio of 1:10; retaining and concentrating a filtrate; spray-drying concentrated thick paste; and then crushing and sieving to obtain the panax notoginseng extract.

A preparation method of a hirudin formula composition with anticoagulant and hypolipidemic effects includes the hirudin formula composition with anticoagulant and hypolipidemic effects and the following steps:
step 1: weighing formulated amounts of hirudin polypeptide, red yeast rice, nattokinase, sulforaphane, agaricus blazei murill, quercetin, hirudin and panax notoginseng extract;
step 2: adding the hirudin polypeptide, the red yeast rice, the nattokinase, the sulforaphane, the agaricus blazei murill, the quercetin, the hirudin and the panax notoginseng extract into a mixer, and mixing well uniformly; and
step 3: preparing the mixture in step 2 into capsules.

### Comparative Example

The blood of a patient with coagulopathy was drawn and subjected to four coagulation tests, and the results were retained; and
the blood of a patient with hyperlipidemia was drawn and subjected to four blood lipid tests, and the results were retained.

### Experimental Example

Three parts of blood of the patient with coagulopathy in the comparative example were drawn, respectively added with the hirudin formula compositions with anticoagulant and hypolipidemic effects in Example 1, Example 2 and Example 3, and then subjected to four coagulation tests; and
three parts of blood of the patient with hyperlipidemia in the comparative example were drawn, respectively added with the hirudin formula compositions with anticoagulant and hypolipidemic effects in Example 1, Example 2 and Example 3, and then subjected to four coagulation tests.

Table 1 shows a performance analysis table of the experimental example and the comparative example for anticoagulant and hypolipidemic effects.

TABLE 1

| | | Examples | | | |
|---|---|---|---|---|---|
| | | Experimental example | | | Comparative |
| Items | | Example 1 | Example 2 | Example 3 | example |
| Four | Activated partial | Normal | Normal | Normal | Low |
| coagulation | thromboplastin time | | | | |
| tests | Prothrombin time | Normal | Normal | Normal | Low |
| | Thrombin time | Normal | Normal | Normal | Low |
| | Plasma fibrinogen | Normal | Normal | Normal | Low |
| Four blood | Total cholesterol | Normal | Normal | Normal | High |
| lipid tests | Triglyceride | Normal | Normal | Normal | High |
| | High-density | Normal | Normal | Normal | High |
| | lipoprotein cholesterol | | | | |
| | Low-density | Normal | Normal | Normal | High |
| | lipoprotein cholesterol | | | | |

The examples of the disclosure are described in detail above. However, the disclosure is not limited to the above-mentioned examples, and various changes can be made without departing from the purpose of the disclosure within the scope of knowledge possessed by those of ordinary skill in the art.

What is claimed is:

1. A hirudin formula composition with anticoagulant and hypolipidemic effects, comprising the following components by weight:

| | |
|---|---|
| hirudin polypeptide | 50 mg to 500 mg; |
| red yeast rice | 10 mg to 100 mg; |
| nattokinase | 10 mg to 100 mg; |
| sulforaphane | 10 mg to 100 mg; |
| agaricus blazei murill | 10 mg to 100 mg; |
| quercetin | 10 mg to 100 mg; |
| hirudin | 10 mg to 300 mg; and |
| panax notoginseng extract | 50 mg to 300 mg. |

2. The hirudin formula composition with anticoagulant and hypolipidemic effects according to claim 1, wherein a preparation method of the hirudin polypeptide comprises: homogenizing fresh leeches in water, wherein a weight ratio of the water to the leeches is (1 to 3):1; centrifuging the homogenate at 10000 r/min for 10 min, and then filtering a precipitate; mixing the precipitate and water in a reactor in a dosage ratio of (3 to 15 g): 100 ml; controlling a pH value to 7.0 to 10.0, and then adding alkaline protease; reacting for 1 to 5 h at a reaction temperature of 40 to 70° C. and an oscillation speed of 100 to 300 rpm; and freeze-drying to obtain the hirudin polypeptide.

3. The hirudin formula composition with anticoagulant and hypolipidemic effects according to claim 1, wherein a preparation method of the nattokinase comprises: inoculating a strain, that is isolated and screened from natto, into a liquid medium, culturing at a constant temperature of 37° C., and centrifuging at 4000 rpm for 30 min; taking a supernatant, i.e., a crude enzyme solution, and removing impure proteins and polysaccharides from the crude enzyme solution; taking a supernatant, adding ammonium sulfate slowly to the supernatant to a saturation of 25%, keeping at low temperature overnight, and centrifuging at 4000 rpm for 30 min to remove impure proteins; then taking a supernatant, adding ammonium sulfate to the supernatant until a final saturation is 60%, keeping at low temperature overnight, and centrifuging at 4000 rpm for 30 min to remove polysaccharides; and collecting a precipitate, dissolving the precipitate in a buffer solution, desalting by dialysis, removing ammonium sulfate, and purifying a sample to obtain the nattokinase.

4. The hirudin formula composition with anticoagulant and hypolipidemic effects according to claim 3, wherein the method for removing the impure proteins and the polysaccharides from the crude enzyme solution is performed by fractional precipitation of ammonium sulfate, and the purification process comprises column chromatography, dialysis, and membrane separation.

5. The hirudin formula composition with anticoagulant and hypolipidemic effects according to claim 1, wherein an extraction step of the sulforaphane comprises: taking cruciferous vegetables, adding 8 to 15 times the amount of water, and performing enzymatic hydrolysis by heating in a water bath at a temperature of 30° C. to 45° C. for 2 to 6 h; and then, adding methanol for ultrasonic extraction, centrifuging the obtained extract to remove a supernatant, and freeze-drying to obtain the sulforaphane.

6. The hirudin formula composition with anticoagulant and hypolipidemic effects according to claim 1, wherein a preparation method of the quercetin comprises: crushing oak barks, extracting with 8 to 12 times the amount of ethanol or methanol, and then filtering, concentrating and crystallizing to obtain a crude quercetin product; and finally performing further separation and purification to obtain the high-purity quercetin.

7. The hirudin formula composition with anticoagulant and hypolipidemic effects according to claim 1, wherein an extraction step of the hirudin comprises: taking fresh leeches, washing impurities and bacteria on the surfaces of the fresh leeches, and chopping the fresh leeches into fine particles; putting the chopped leeches into 10 to 15 times the amount of purified water, and stirring them in a thermostatic shaker for 1 h; centrifuging the stirred liquid, collecting a precipitate, and washing the precipitate with purified water; repeating this step for three times until a pure leech extract is collected; and drying the leech extract through a nitrogen sampler to obtain a dried hirudin polypeptide.

8. The hirudin formula composition with anticoagulant and hypolipidemic effects according to claim 1, wherein a preparation method of the panax notoginseng extract comprises: crushing panax notoginseng, and pouring into an extraction tank; extracting the panax notoginseng with clear water according to a mass ratio of 1:(10 to 15); retaining and concentrating a filtrate; spray-drying concentrated thick paste; and then crushing and sieving to obtain the panax notoginseng extract.

9. A preparation method of a hirudin formula composition with anticoagulant and hypolipidemic effects, comprising the hirudin formula composition with anticoagulant and hypolipidemic effects according to claim 1 and the following steps:
step 1: weighing formulated amounts of hirudin polypeptide, red yeast rice, nattokinase, sulforaphane, agaricus blazei murill, quercetin, hirudin and panax notoginseng extract;
step 2: adding the hirudin polypeptide, the red yeast rice, the nattokinase, the sulforaphane, the agaricus blazei murill, the quercetin, the hirudin and the panax notoginseng extract into a mixer, and mixing well uniformly; and
step 3: preparing the mixture in step 2 into capsules.
